(19) United States
(12) Patent Application Publication
(10) Pub. No.: US 2023/0355477 A1
(43) Pub. Date: Nov. 9, 2023

(54) BLISTER CONTENTS ACCOMMODATION DEVICE, DRUG ADMINISTRATION MANAGEMENT SYSTEM INCLUDING SAME, AND METHOD FOR DETERMINING WHETHER CONTENTS ARE DISCHARGED

(71) Applicant: COLEDY INC., Seoul (KR)

(72) Inventor: Kyung Do PARK, Seoul (KR)

(21) Appl. No.: 18/013,652

(22) PCT Filed: Jun. 30, 2021
(86) PCT No.: PCT/KR2021/008245

§ 371 Date: Dec. 29, 2022

(30) Foreign Application Priority Data
Jun. 30, 2020 (KR) 10-20-20-0080590

(51) Int. Cl. A61J 7/00; A61J 7/02

(52) U.S. Cl. CPC A61J 7/0069; A61J 7/0076; A61J 7/02; A61J 2200/70

(57) ABSTRACT

The present disclosure relates to a contents accommodation device for blister packs that is capable of detecting discharge of contents, a medication management system including the same, and a method of determining whether contents are discharged. The contents accommodation device according to the present disclosure includes a blister seating portion where a blister in which contents are packed is seated, a container main body configured to accommodate the blister seating portion in an upper portion thereof and having a space at a lower portion to accommodate contents that fall after being extruded from the blister, and a passing portion configured to discharge the contents moving thereto from the container main body to an outside, wherein the passing portion includes a passage portion configured to serve as a movement passage of the contents and a detecting portion configured to detect movement of the contents, an opening is formed at one end of the passage portion, and the contents are discharged to the outside through the opening at the one end of the passage portion after entering the passage portion from the container main body.

### TECHNICAL FIELD

The present disclosure relates to a blister-packed contents accommodation device that is capable of detecting discharge of contents, and more particularly, to a passing device for contents in a blister pack and an accommodation device having the same, a medication management system including the same, and a method of determining whether contents are discharged which are capable of accurately detecting contents discharged from the accommodation device and, based on the detection, allowing a user to take the contents in fixed amounts and manage medication.

### BACKGROUND ART

Generally, blister packaging is a type of packaging in which a plastic sheet is thermoformed to form one or more concave portions, contents are placed therein, an opening is covered with a film or an aluminum foil or the like, and a peripheral portion is adhered to a base material. The use of blister packaging is a global trend for prevention of drug misuse and drug preparation errors as well as for sanitary and safety reasons. Among different types of blister packaging, Press-Through-Pack (PTP) packaging, which is a form of packaging pressed with a finger to take out contents, is used for various objects such as drugs and health supplements and is evaluated as having excellent portability due to individually packaging each tablet and evaluated as being optimal for packaging small tablets due to being difficult to be degraded.

Meanwhile, according to a study published in 2018, among new diabetic patients in South Korea with low adherence to medication during the first two years, the mortality rate increased up to 45%, and the incidence rate of cerebrovascular disease increased up to 41%. In order to prevent overuse or addiction to contents such as drugs or health supplements, as well as to maximize the efficacy thereof, it is essential for the contents to be taken in fixed amounts. Also, there is a need to manage medication so that the contents are taken in fixed amounts through accurate detection of an amount of the contents discharged.

### DISCLOSURE

### Technical Problem

The present disclosure provides a contents passing device, an accommodation device, and a medication management system which are capable of accurately detecting contents discharged from the accommodation device accommodating the contents therein and, based on the detection, allowing a user to take the contents in fixed amounts and manage medication.

The present disclosure also provides a passing device and an accommodation device which are capable of accurately distinguishing between a state in which contents are stuck in the passing device and a state in which the contents are discharged therefrom, thus automatically detecting the discharge of the contents.

### Technical Solution

The present disclosure provides a contents accommodation device including: a blister seating portion where a blister pack in which contents are packed is seated; a container main body configured to accommodate the blister seating portion in an upper portion thereof and having a space at a lower portion to accommodate contents that fall after being extruded from the blister pack; and a passing portion configured to discharge the contents moving thereto from the container main body to an outside, wherein the passing portion includes a passage portion configured to serve as a movement passage of the contents and a detecting portion configured to detect movement of the contents, an opening is formed at one end of the passage portion, and the contents are discharged to the outside through the opening at the one end of the passage portion after entering the passage portion from the container main body.

Also, the passing portion of the contents accommodation device may be connected to one side of the container main body, the passage portion may form a movement path to discharge the contents in fixed amounts and may communicate via an inlet with the space of the container main body that accommodates the contents, the container main body may include a guide passage disposed adjacent to the inlet to guide the contents toward the passage portion, and the contents may enter the passage portion along the guide passage.

The detecting portion may include a light emitting portion and a light receiving portion disposed to face the light emitting portion, light radiated from the light emitting portion may be detected by the light receiving portion after passing through the path along which the contents move to be discharged, whether the contents are discharged may be determined by a quantity of light detected by the light receiving portion, and the contents may be determined as having been discharged in a case in which the quantity of light detected by the light receiving portion that belongs to a standard light quantity range becomes a predetermined reference light quantity or less and then rises back into the standard light quantity range.

Also, the light receiving portion may include a first light receiving portion disposed at an upstream side of the path along which the contents move to be discharged and a second light receiving portion disposed at a downstream side of the path, the light emitting portion may include one or more light emitting portions disposed to face the light receiving portion, and whether the contents are discharged may be determined based on a light quantity change pattern by time that is detected by each of the first light receiving portion and the second light receiving portion.

Also, the contents may be determined as not having been discharged in a case in which the contents are determined as having passed through an area of the second light receiving portion prior to passing through an area of the first light receiving portion.

A first point in time at which a quantity of light detected by the first light receiving portion that belongs to a first standard light quantity range becomes a first reference light quantity or less and then rises back into the first standard light quantity range is determined as a point in time at which the contents pass through the area of the first light receiving portion. A second point in time at which a quantity of light detected by the second light receiving portion that belongs to a second standard light quantity range becomes a second reference light quantity or less and then rises back into the second standard light quantity range is determined as a point in time at which the contents pass through the area of the second light receiving portion. The first standard light quantity range and the second standard light quantity range are ranges of a quantity of light detected by each light receiving portion in a state in which the contents do not pass. The contents are determined as having been normally discharged in a case in which the first point in time is the same as or earlier than the second point in time.

The blister seating portion may be installed between a container lid and the container main body and seated on an upper end of the container main body and may include a seating plate and a seating lid (233) coupled to an upper portion of the seating plate, the seating lid may be hinge-coupled to one side of the seating plate, the seating plate may support the blister pack from below, the seating lid may be a plate in which a plurality of through-holes aligned with each other are formed, and contents accommodating portions of the blister pack seated on the seating plate may each be disposed in the through-holes.

Also, the guide passage may be formed to be bent from the inlet, the passage portion may be formed to have a slope from the space, and a width of the passage portion may be formed to allow only one content to pass at a time.

Also, a signal detected by the detecting portion may be processed by an information processing device included in the passing device or may be processed by a separate external device.

According to another aspect of the present disclosure, a plurality of support portions may be disposed to be aligned with each other inside the container main body to correspond to a contents arrangement structure of the blister pack. Upon a user pressing one content, the plurality of support portions may support portions of the blister at both sides of the pressed content from below to facilitate extrusion of the content.

The present disclosure also provides a medication management system including a contents accommodation device, a medication management server, and a medication guide terminal, wherein, in the medication management system, the medication guide terminal may include an output portion and a medication guide terminal controller configured to receive medication state information from a detecting portion, receive medication schedule information from the medication management server, generate medication counseling information for a user by using the medication schedule information and the medication state information, and then output the medication counseling information through the output portion. Also, the output portion of the medication management system may output the medication counseling information using a video or audio medium.

The present disclosure also provides a method of determining whether contents are discharged based on a quantity of light detected by a detecting portion of a contents accommodation device. The method includes; a detection step in which both a first light receiving portion and a second light receiving portion detect a quantity of light that deviates from a standard light quantity range and is lower than a reference light quantity; a detection step in which the first light receiving portion detects a quantity of light that deviates from a first reference light quantity and belongs to a first standard light quantity range, and the second light receiving portion detects a quantity of light that deviates from a second reference light quantity and belongs to a second standard light quantity range; a comparison step in which a first point in time, at which the quantity of light detected by the first light receiving portion that belongs to the first standard light quantity range becomes the first reference light quantity or less and then rises back into the first standard light quantity range, and a second point in time, at which the quantity of light detected by the second light receiving portion that belongs to the second standard light quantity range becomes the second reference light quantity or less and then rises back into the second standard light quantity range, are compared; and a determination step in which the contents are determined as having been discharged in a case in which the first point in time is the same as or earlier than the second point in time.

### Advantageous Effects

According to one aspect of the present disclosure, a passing device with high accuracy of detecting discharge of contents packed in blister packaging, in particular, Press-Through-Pack (PTP) packaging, can be provided, and based on the passing device, a user can manage medication so that the contents are taken in fixed amounts.

According to another aspect of the present disclosure, by providing a passing device for blisters and an accommodation device having the same that are simple and can accurately determine whether contents are discharged in fixed amounts, it is possible to provide a fixed amount passing device that is both accurate and economically feasible.

### DESCRIPTION OF DRAWINGS

FIG. 1 is a perspective view of an assembled state of a contents accommodation device having a passing device according to an embodiment of the present disclosure, a perspective view of a state in which a lid is open, a perspective view of a state in which a blister seating portion is separated, a perspective view of a state in which a contents lid plate is open, and a perspective view of a state in which the blister seating portion and a blister are separated, respectively.

FIG. 2 is a conceptual diagram illustrating a contents discharge process of the contents accommodation device according to an embodiment of the present disclosure from a side.

FIG. 3 is a cross-sectional view of the contents accommodation device having the passing device according to an embodiment of the present disclosure.

FIG. 4 is a block diagram of a detecting portion configured to detect movement of contents according to an embodiment of the present disclosure.

FIG. 5 is a view showing graphs illustrating a quantity of light measured by the detecting portion configured to detect movement of contents in the passing device according to the present disclosure.

FIG. 6 is a view illustrating light quantities with time in a case in which pills are discharged to the outside via a passage portion in one embodiment of the present disclosure.

FIG. 7 is a view illustrating light quantities with time in a case in which a user or the like arbitrarily injects contents from the outside, in one embodiment of the present disclosure.

FIG. 8 is a block diagram of a medication management system including a medication guide terminal, a medication management server, and a contents accommodation device which has a passing device according to the present disclosure.

FIG. 9 is a perspective view of an assembled state of a contents accommodation device having a passing device according to another embodiment of the present disclosure.

FIG. 10 is a perspective view of a state in which a blister is seated on the contents accommodation device having the passing device according to another embodiment of the present disclosure.

### MODES OF THE INVENTION

Hereinafter, a passing device, an accommodation device, a medication management system, and a discharge detection method according to one embodiment of the present disclosure will be described with reference to the accompanying drawings. In this process, thicknesses of lines, sizes of elements, or the like that are illustrated in the drawings may have been exaggerated for clarity and convenience of description. Also, the terms used herein are terms defined in consideration of functions in the present disclosure and may vary according to an intention or practice of a user or an operator. Therefore, the terms should be defined on the basis of the content throughout the specification.

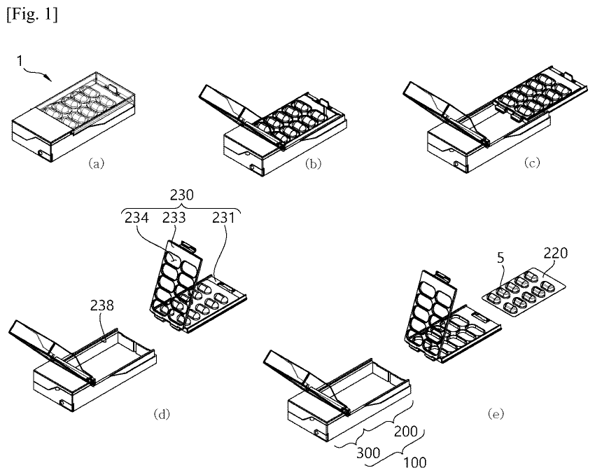

FIGS. 1A to 1E are a perspective view of an assembled state of a contents accommodation device having a passing device according to an embodiment of the present disclosure, a perspective view of a state in which a lid is open, a perspective view of a state in which a blister seating portion is separated, a perspective view of a state in which a contents lid plate is open, and a perspective view of a state in which the blister seating portion and a blister are separated, respectively.

As illustrated in FIG. 1, a contents accommodation device 1 according to an embodiment of the present disclosure includes a blister seating portion 230 and a housing 100. The blister seating portion 230 is a portion where a blister 220 in which contents 5 are packed is seated and stored. The blister seating portion 230 is detachably coupled to the housing 100. The housing 100 is a container configured to accommodate or store the contents 5 and includes a container main body 200 configured to deliver the contents 5 received from the blister 220 to a passing portion 300 and the passing portion 300 configured to discharge the contents 5 received from the container main body 200 to an outside and detect whether the contents 5 are discharged.

The container main body 200 may accommodate the blister seating portion 230 in an upper portion thereof and have a space at a lower portion to accommodate the contents 5 that fall after being extruded from the blister 220. Also, the passing portion 300 may be formed to extend from one end of the container main body 200, and the passing portion 300 may discharge the contents 5 sliding due to gravity from the container main body 200 to the outside. The passing portion 300 includes a passage portion, which serves as a movement passage of the contents 5, and a passing device, and an opening through which the contents 5 may be discharged to the outside and an opening member configured to open or close the opening are included at one surface of the passing portion 300. Here, the passing portion 300 and the container main body may be separately formed or integrally formed. Also, the passing portion 300 and the container main body communicate via an inlet.

The housing 100 may include a passing portion lid and a container lid. The passing portion lid and the container lid may be coupled to an upper portion of the housing 100. The container lid may be hinge-coupled to one side of an upper end of the container main body 200 to be opened or closed. The passing portion lid and the container lid may be separately formed.

The blister seating portion 230 may be installed between the container main body and the container lid and seated on the upper end of the container main body 200. Here, a groove 238 may be formed at the upper end of the container main body and may fix and support the blister seating portion 230 to allow the blister seating portion 230 to be seated on the upper end of the container. Also, the blister seating portion 230 may be slidably coupled along the groove 238 at the upper end of the container main body and be detachable from the container main body.

The blister seating portion 230 includes a seating plate 231 and a seating lid 233 coupled to an upper portion of the seating plate 231. The seating lid 233 may be hinge-coupled to one side of the seating plate 231 to be opened or closed, and the seating plate 231 may support the blister 220 from below so that the blister 220 may be seated and fixed between the seating lid 233 and the seating plate 231. The seating lid 233 may be a plate in which a plurality of through-holes 234 aligned with each other are formed, and contents 5 accommodating portions of the blister seated on the seating plate 231 may each be disposed in the through-holes 234.

Here, the blister 220 may be Press-Through-Pack (PTP) packaging, and the contents 5 may be solid. Also, the contents 5 may be food and/or drugs, such as pills (tablets), that may be packed in the blister 220. The contents 5 are seated on the blister seating portion 230 while packed in the blister 220.

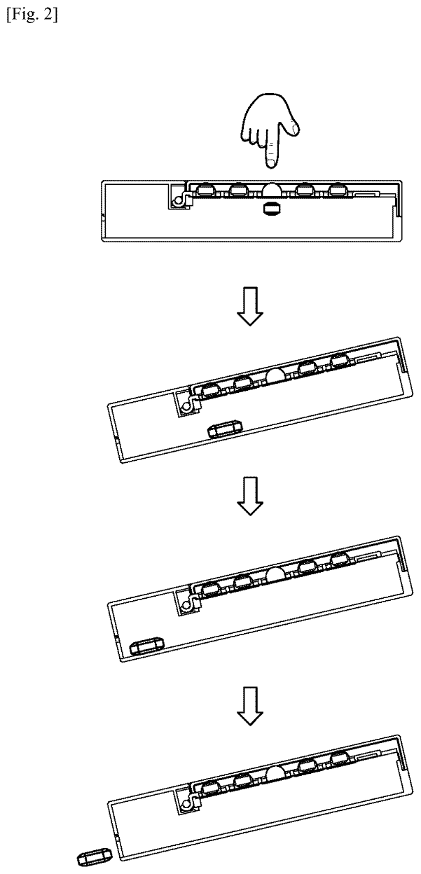

FIG. 2 is a conceptual diagram illustrating a contents discharge method of the contents accommodation device according to an embodiment of the present disclosure from a side.

According to an embodiment of the present disclosure, the contents may be pressed from the contents accommodating portions of the blister by a user and fall to the container main body, may enter the passage portion from the container main body, and then be discharged to the outside through the opening. The contents discharge method of the contents accommodation device may be classified into four operations including a contents pressing operation, a passing portion entry operation, a discharge detection operation, and an external discharge operation.

The first operation is the contents pressing operation which is an operation in which the contents packed in the blister are pressed by a user and delivered to the container main body. The position of the blister may be fixed by the plurality of through-holes 234 formed in the seating lid 233. Here, when the contents accommodating portions of the blister seated on the seating plate 231 are pressed, the contents are extruded from the blister and fall onto a bottom of the container main body coupled to the blister seating portion. The container main body may accommodate the contents discharged from the blister and deliver the contents to the passing portion.

The second operation is the passing portion entry operation which is an operation in which the contents enter the passing portion due to gravity in a state in which the contents accommodation device is tilted at a predetermined angle or more. The container main body communicates with the passage portion of the passing portion via the inlet, and a guide passage is disposed adjacent to the inlet in the container main body. Therefore, when the user tilts the container accommodation device which is in a horizontal state, the contents on the bottom of the container main body move to the passing portion along the guide passage. Here, the contents accommodation device may be tilted downward, which is the direction of gravity and a direction toward where the passing portions is disposed, and due to the contents accommodation device being tilted or shaken, gravitational motion of the contents or transfer of kinetic force of the contents to the contents accommodation device causes the contents to enter the passing portion.

The third operation is the discharge detection operation which is an operation in which the contents delivered to the passing portion through the passing portion entry operation are detected by the passing device included in the passing portion. The passing portion includes the passage portion and the passing device, the passing device includes a control module, a communication module, and a detecting portion, and the detecting portion includes a light emitting portion, a light receiving portion, and a sensing controller. Here, the detecting portion may detect movement of the contents. Details thereof will be described below.

The fourth operation is the external discharge operation which is an operation in which the contents detected as being discharged pass through the opening formed at one end of the passage portion and are discharged from the contents accommodation device to the outside.

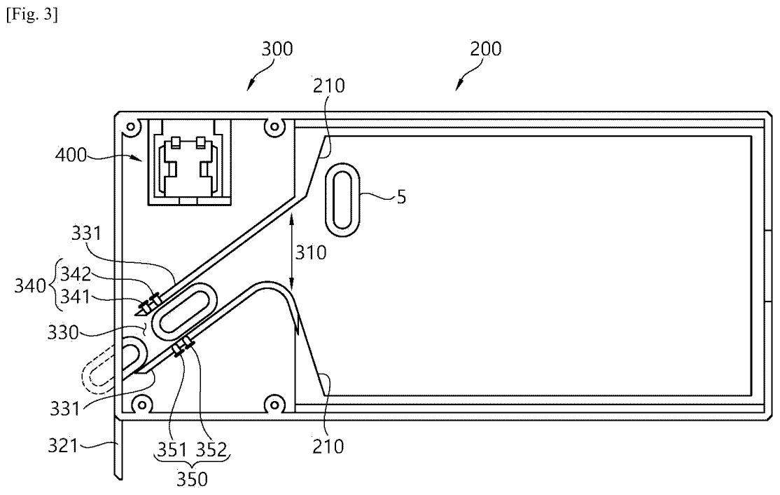

FIG. 3 is a cross-sectional view of the contents accommodation device having the passing device according to an embodiment of the present disclosure.

The container main body and the passing portion 300 of the contents accommodation device are disclosed in detail in FIG. 3.

Referring to FIG. 3, a passage portion 330 in the form of a duct forming a movement path for discharging the contents 5 in fixed amounts is formed in the passing portion 300, and the passage portion 330 communicates via an inlet 310 with the space of the container main body that accommodates the contents 5. Also, the container main body includes a guide passage 210 disposed adjacent to the inlet 310 to guide the contents 5 to the passage portion 330. Here, the contents 5 in the space of the container main body that accommodates the contents 5 may enter the passage portion 330 along the guide passage 210. The guide passage 210 is formed to be bent from the inlet 310, and the passage portion 330 is formed to have a slope from the space accommodating the contents 5. Here, a width of the passage portion 330 is formed to allow only one content 5 to pass at a time. Also, the passage portion 330 is formed so that the inlet 310, the passage portion 330, and the opening are connected in this order in a substantially straight line.

Specifically, the passage portion 330 is formed by an extending sidewall 331, one end of the passage portion 330 is an opening communicating with the outside, and the other end is connected to the guide passage 210 of the container main body. The guide passage 210 may be formed in the shape of a funnel which has a width gradually narrowing toward the inlet 310 to facilitate entry of the contents 5 into the passage portion 330. Therefore, in a case in which the contents accommodation device is tilted, the contents 5 may slide from the container main body along the movement path, including the inlet 310 and the passage portion 330 in this order, along the guide passage 210.

The size of the inlet 310 and the internal cross-sectional area, size, and angle of the passage portion 330 may be designed according to the size and shape of the contents 5 that are accommodated in the contents accommodation device 1 and pass through the passage portion 330. Specifically, in a case in which a length of one end portion of one content 5 passing through the passage portion 330 is different from a length of the other end portion thereof, since a passage width minimum inner diameter, which is a minimum inner diameter length in an inner space of the passage portion 330, is less than or equal to two times a maximum outer diameter in a short side direction of the content 5, which is a maximum outer diameter length in the short side direction of one content 5, two or more contents 5 are not able to simultaneously enter or pass through the passage portion 330, and only one content 5 is able to enter or pass through the passage portion 330 at a time. Also, in the case in which the length of one end portion of one content 5 passing through the passage portion 330 is different from the length of the other end portion thereof, the passage portion 330 guides the contents 5 so that the contents 5 pass through the passage portion 330 in a longitudinal direction of a longer end portion. This is to discharge the contents 5 in fixed amounts and improve accuracy of detecting the discharge of the contents 5, and the passage portion 330 may longitudinally extend with a predetermined angle. Here, “fixed amount” refers to the same number or amount or the amount or number varying within an error range.

The size of the opening may be designed according to the size and shape of the contents 5. By an opening member 321 blocking the opening in a general state in which the contents 5 are stored, other than a state in which the contents 5 are discharged, foreign substances such as air or dust are suppressed from entering the housing through the opening. Also, the opening member 321 may be slidably coupled to the opening.

Meanwhile, the method of detecting the discharge of the contents from the passing portion 300 is performed through the passing device. Here, the passing device includes a control module 400, a communication module, and a detecting portion, and the detecting portion includes at least one light emitting portion 350 configured to emit light, at least one light receiving portion 340 disposed to face the light emitting portion 350 and configured to receive the light emitted from the light emitting portion 350, and a sensing controller. Specifically, whether the contents 5 are discharged is determined by a quantity of light detected by the light receiving portion 340 after light radiated from the light emitting portion 350 passes through a path, along which the contents 5 move to be discharged, and is detected by the light receiving portion 340. In a case in which the contents 5 pass, the light radiated from the light emitting portion 350 is blocked by the contents 5, and a small quantity of light is detected temporarily. Therefore, the detecting portion may determine that the contents 5 have been discharged in a case in which the quantity of light detected by the light receiving portion 340 that belongs to a standard light quantity range becomes a predetermined reference light quantity or less and then rises back into the standard light quantity range.

The light emitting portion 350 radiates light toward the contents 5, and the light receiving portion 340 receives the light radiated from the light emitting portion 350. For example, the light emitting portion 350 may be a light emitting diode (LED), and the light receiving portion 340 may be a photodiode. The sensing controller controls the light emitting portion 350 and the light receiving portion 340 and processes a light signal emitted from the light emitting portion 350 and then received by the light receiving portion 340. The sensing controller may be implemented using a microcontroller unit (MCU), a field programmable gate array (FPGA), or the like and may be physically included in a printed circuit board (PCB). Also, the light emitting portion 350 and the light receiving portion 340 may be physically and electrically connected to the sensing controller (e.g., PCB) and fixed. In addition, the light emitting portion 350 and the light receiving portion 340 may be disposed to face each other at one side of the sidewall 331 constituting the passage portion 330, and a transmission wall through which light may be transmitted may be disposed between the passage portion 330 and a through-hole formed in the sidewall 331 of the passage portion 330.

In order to face the light emitting portion 350, the light receiving portion 340 is disposed at the other side of the sidewall 331 forming the passage portion 330. Like the light emitting portion 350, the light receiving portion 340 may be disposed so that two photodiodes are disposed to be aligned in a row at the through-hole formed in the sidewall 331, and a transmission wall through which light may pass may be disposed between the passage portion 330 and the through-hole.

In addition, the passing device or the contents accommodation device having the passing device may include a motion sensor. Accordingly, the detecting portion may not operate in the general state in which the contents 5 are stored, and power loss can be reduced.

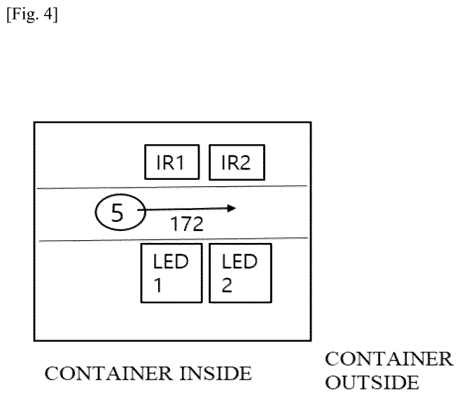

FIG. 4 is a block diagram of the detecting portion configured to detect movement of contents according to an embodiment of the present disclosure.

Referring to FIG. 4, as the light emitting portions 350, two LEDs, or infrared LEDs, are arranged in a row along the passage portion. As the light receiving portions (IR1, IR2), photodiodes that can detect infrared rays are arranged in a row at positions facing the two LEDs along a path of the passage portion. The light receiving portion 340 is not limited to being a photodiode and may be any other photosensitive element that can sense light.

The light receiving portion 340 includes a first light receiving portion (IR1) 342 disposed at an upstream side of the path along which the contents move to be discharged and a second light receiving portion (IR2) 341 disposed at a downstream side of the path.

The light emitting portion 350 consists of one or more light emitting elements disposed to face the light receiving portion 340 and may consist of two light emitting elements. Both of the two light emitting elements may be turned on and radiate light toward the passage portion, but only one of the two may be turned on. As an alternative, a single light emitting element may face two light receiving portions 340 and may be disposed in between the two light receiving portions 340 while being at an equal distance from the two light receiving portions 340. Light radiated from the light emitting portion 350 crosses the passage portion and is detected by the light receiving portion 340.

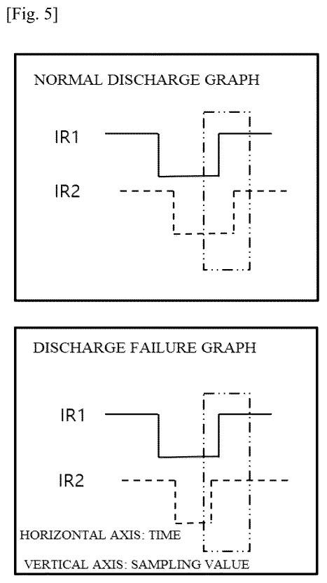

FIG. 5 is a view showing graphs illustrating a quantity of light measured by the detecting portion configured to detect movement of contents in the passing device according to the present disclosure.

Patterns of changes in the quantity of light measured by the detecting portion having two light receiving portions configured to detect movement of contents according to the present disclosure are disclosed by the graphs in FIG. 5.

A quantity of light detected by each of the two light receiving portions (IR1, IR2) is an analog value, and an analog-to-digital converter (ADC) converts the analog value into a digital value to obtain a sampling value. The sensing controller of the detecting portion may use the sampling value to detect whether a pill passes. In a case in which a portion between the light emitting portion and the light receiving portion is blocked by a pill, since the quantity of received light decreases, the sampling value also decreases at the moment the pill passes.

Referring to FIG. 5, the horizontal axis represents time, and the vertical axis represents a sampling value obtained by the ADC converting the quantity of light detected by the light receiving portions (IR1, IR2) into a digital value. It is possible to determine success or failure of discharge by comparing waveforms by time of the quantities of light detected by the light receiving portions (IR1, IR2) at the time of success and failure of discharge. Such a determining method addresses a conventional problem of about 5% of determination failure in a case in which whether discharge has occurred is determined based on changes in the quantity of light detected by a single light receiving portion and makes a determination error close to 0. The quantity of light detected by the first light receiving portion is shown in blue, and the quantity of light detected by the second light receiving portion is shown in red. Also in other drawings described below, in the same manner, the quantity of light detected by the first light receiving portion is shown in blue, and the quantity of light detected by the second light receiving portion is shown in red.

Specifically, the contents are determined as having passed through an area of the first light receiving portion in a case in which a quantity of light detected by the first light receiving portion (IR1) that belongs to a first standard light quantity range becomes a first reference light quantity or less and then rises back into the first standard light quantity range. The contents are determined as having passed through an area of the second light receiving portion in a case in which a quantity of light detected by the second light receiving portion (IR2) that belongs to a second standard light quantity range becomes a second reference light quantity or less and then rises back into the second standard light quantity range. Therefore, whether the contents are discharged may be determined based on patterns of changes in the quantity of light with time detected by each of the first light receiving portion and the second light receiving portion.

The first standard light quantity range and the second standard light quantity range are ranges of a quantity of light detected by each light receiving portion in a state in which the contents do not pass, and the contents are determined as having been normally discharged in a case in which the contents are determined as having passed through the area of the first light receiving portion prior to or while passing through the area of the second light receiving portion. Otherwise, that is, in a case in which the contents pass through the area of the first light receiving portion after passing through the area of the second light receiving portion, a failure of discharge is determined as having occurred. That is, the contents are determined as not having been discharged in a case in which the contents are determined as having passed through the area of the second light receiving portion prior to passing through the area of the first light receiving portion.

Also, a first point in time at which the quantity of light detected by the first light receiving portion reaches a downward peak and then reaches the first standard light quantity range and a second point in time at which the quantity of light detected by the second light receiving portion reaches a downward peak and then reaches a second reference value again may be compared, and the contents may be determined as having been discharged in a case in which the first point in time is the same as or earlier than the second point in time.

The above determination process may be executed by the sensing controller included in the detecting portion of the passing device or executed by a processor or the like included in the accommodation device but is not limited thereto. An analog value of the detected light quantity may be converted into a digital value by the ADC, and the digital value may be sent to an external server or terminal by a wired/wireless communication module for the external terminal or server to execute the determination process. The external terminal or server may be a medication management terminal or medication management server. The inventors of the present disclosure have obtained the following specific experimental results by repeating an experiment multiple times in order to verify light quantity detection by the two detecting portions shown by the graphs in FIG. 5.

As can be seen from comparing the graphs in FIG. 5, as compared to time T1 during which a detected light quantity continues to be a predetermined reference light quantity or less in the case of a success of discharge, a predetermined amount of time T2 during which a detected light quantity continues to be the predetermined reference light quantity or less in the case of a failure of discharge is larger. The predetermined amount of time T2 is two times or larger than the time T1 during which a detected light quantity continues to be the reference light quantity or less in the case of a success of discharge.

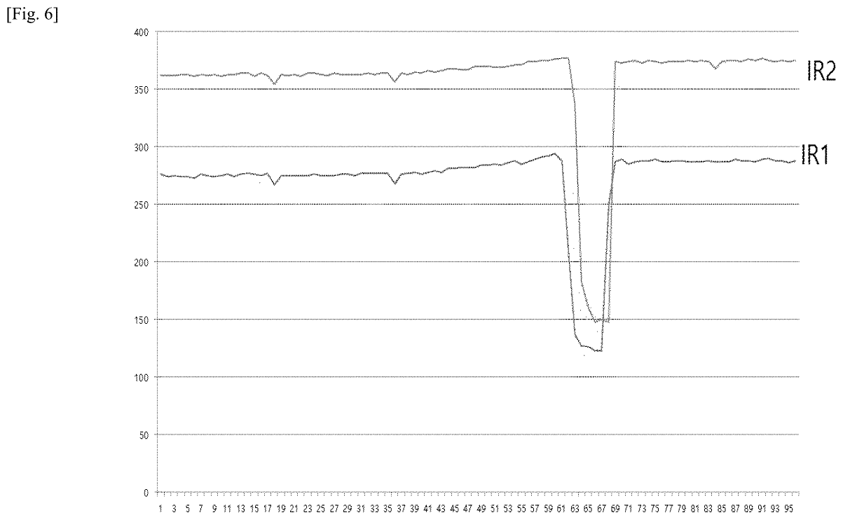

FIG. 6 is a view illustrating light quantities with time in a case in which pills are discharged to the outside via the passage portion in an embodiment of the present disclosure.

Changes in light quantities in a case in which pills are discharged to the outside via the passage portion when the accommodation device having the passing device, in which the two light emitting elements are disposed to be aligned with each other, according to an embodiment of the present disclosure is tilted are disclosed in FIG. 6.

Referring to FIG. 6, the quantity of light detected by the first light receiving portion (IR1) shown in blue is first maintained almost constant within a first standard light quantity range (for example, 250 to 300) due to no passage of contents. Then, as the accommodation device is tilted, the contents enter the passage portion, pass through the areas of the first and second light receiving portions, and are discharged to the outside. Accordingly, the quantity of light becomes a first reference light quantity or less and then rises back into the first standard light quantity range.

Meanwhile, the quantity of light detected by the second light receiving portion (IR2) disposed at a portion of the passage portion closer to the outside of the container first belongs to a second standard light quantity range which is between 350 to 400, becomes a second reference light quantity (in this case, 200) or less, and then rises back into the second standard light quantity range.

Here, in the graph, the time at which the quantity of light detected by the first light receiving portion is recovered and rises back into the first standard light quantity range is the same as or slightly earlier than the time at which the quantity of light detected by the second light receiving portion is recovered and rises back into the second standard light quantity range. Such waveforms of quantities of light are observed in the case of a success of discharge of the contents.

That is, the first point in time, at which the quantity of light detected by the first light receiving portion that belongs to the first standard light quantity range becomes the first reference light quantity or less and then rises back into the first standard light quantity range, and the second point in time, at which the quantity of light detected by the second light receiving portion that belongs to the second standard light quantity range becomes the second reference light quantity or less and then rises back into the second standard light quantity range, may be compared, and the contents may be determined as having been discharged in a case in which the first point in time is the same as or earlier than the second point in time.

In a discharge determination method according to another embodiment of the present disclosure, contents may be determined as having been discharged in a case in which a quantity of light detected by the light receiving portion that belongs to a standard light quantity range becomes a predetermined reference light quantity or less and then rises back into the standard light quantity range. However, in a case in which the detected light quantity continues to be the predetermined reference light quantity or less for a predetermined amount of time or more, the contents may be determined as being stuck in a passing operation portion and may be determined as not having been discharged.

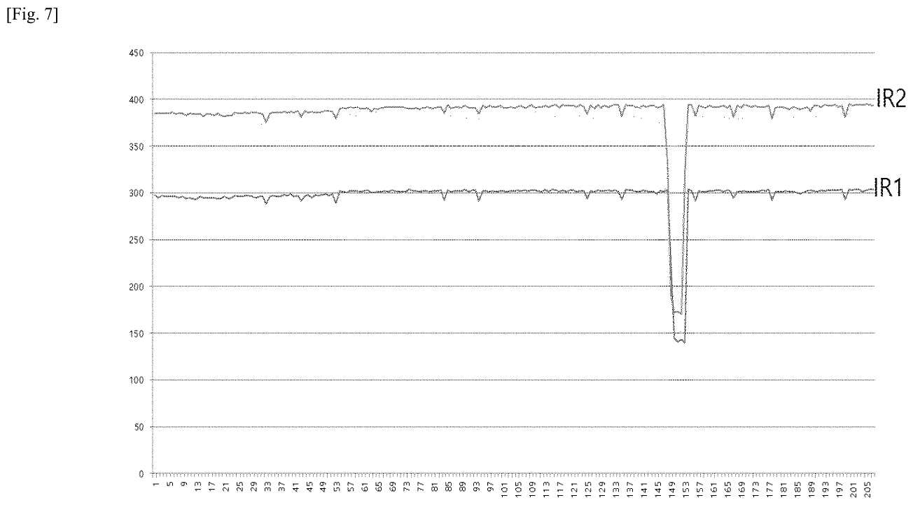

FIG. 7 is a view illustrating light quantities with time in a case in which a user or the like arbitrarily injects contents from the outside, in one embodiment of the present disclosure.

FIG. 7 shows the changes in light quantities in a case in which contents are injected from the outside through the opening in a state in which the accommodation device is tilted to the opposite direction.

Referring to FIG. 7, in the case in which the contents are injected from the outside through the opening, patterns of the discharge of the contents and signals detected by the first and second light receiving portions are opposite. That is, patterns in which the discharge incidentally appears after a downward peak of a signal detected by the first light receiving portion at an inner side of the passage or a downward peak of a signal detected by the second light receiving portion are shown.

Referring to FIG. 7, the quantity of light detected by the first light receiving portion that is shown in blue is first maintained almost constant within the first standard light quantity range (for example, 250 to 300) due to no passage of contents. Then, as the contents are injected through the opening, the contents pass through the areas of the second and first light receiving portions and are accommodated in the housing. Accordingly, the quantity of light becomes the first reference light quantity or less and then rises back into the first standard light quantity range.

Meanwhile, the quantity of light detected by the first light receiving portion (IR1) disposed at a portion of the passage portion closer to the inside of the container belongs to the second standard light quantity range which is between 350 to 400, becomes the second reference light quantity (in this case, 200) or less, and then rises back into the second standard light quantity range.

Here, in the graph, the time at which the quantity of light detected by the second light receiving portion is recovered and rises back into the second standard light quantity range is the same as or slightly earlier than the time at which the quantity of light detected by the first light receiving portion is recovered and rises back into the second standard light quantity range. Such waveforms of quantities of light are observed in the case in which the contents are arbitrarily injected from the outside.

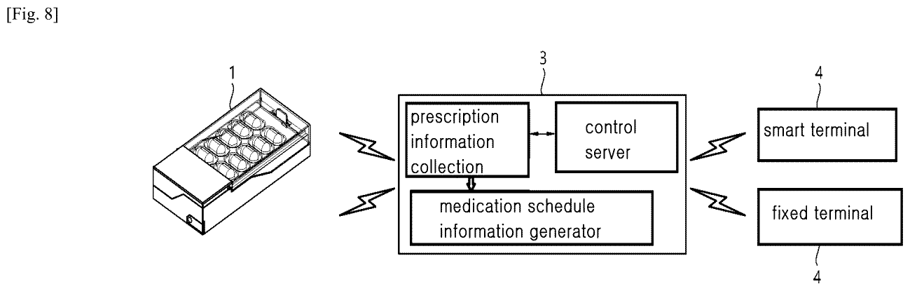

FIG. 8 is a block diagram of a medication management system including a medication guide terminal, a medication management server, and the contents accommodation device which has the passing device according to the present disclosure.

Referring to FIG. 8, the sensing controller of the contents accommodation device 1 may process light quantity signals with time that are detected by the light receiving portions and may generate medication state information according to a result of determining whether the contents have passed. Also, the generated medication state information may be delivered to the medication guide terminal through the control module. Here, the medication state information includes information on contents, information on whether the contents are detected, the detection time at which the contents are detected, and the like. The information on contents may be preset in the sensing controller, the medication management server, or the like, and whether the contents are detected and the detection time may be detected in real time according to whether the contents are detected by the detecting portion.

A medication guide terminal 4 may receive medication state information from the contents accommodation device 1, receive medication schedule information from a medication management server 3, generate medication management information using the received medication schedule information and medication state information, and then output the generated medication management information.

In other words, in the medication management system including the medication management server and the medication guide terminal, the medication guide terminal may include an output portion and a medication guide terminal controller configured to receive medication state information from the detecting portion, receive medication schedule information from the medication management server, generate medication counseling information for a user by using the medication schedule information and the medication state information, and then output the medication counseling information through the output portion, and the output portion may output the medication counseling information using a video or audio medium.

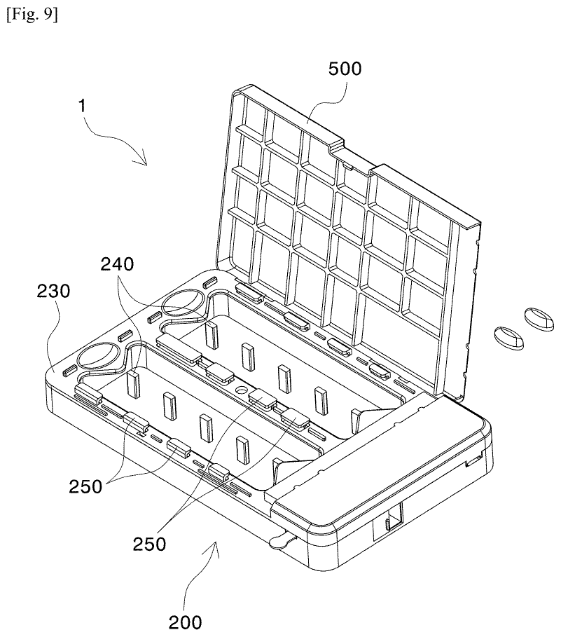

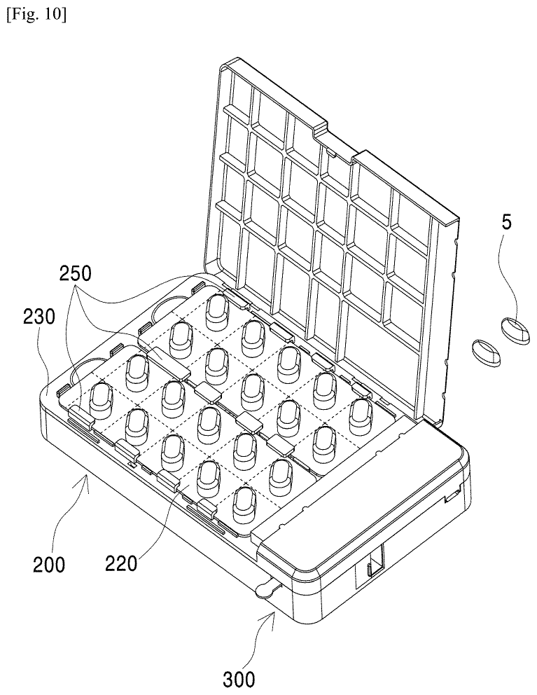

Another embodiment of the present disclosure will be described with reference to FIGS. 9 and 10.

Hereinafter, description of parts identical to those in the embodiments of FIGS. 1 to 8 will be omitted, and only the configurations different therefrom will be described. Therefore, the omitted description may be replaced with the above description relating to FIGS. 1 to 8.

Referring to FIGS. 9 and 10, the housing of the contents accommodation device 1 includes the container main body 200 configured to accommodate the contents 5 that are pressed from the blister 220 and fall downward and the passing portion 300 configured to discharge the contents 5 received from the container main body 200 to the outside and detect whether the contents 5 are discharged. The internal configuration of the passing portion is the same as that illustrated in FIG. 3, and the sensor arrangement and detected signals are the same as those in FIGS. 4 to 8. Thus, descriptions thereof will be omitted.

Referring to FIG. 9, the blister seating portion 230 is a portion where the blister 220 in which the contents 5 are packed is seated and stored. The blister seating portion 230 is disposed at an upper portion of the container main body 200. The blister seating portion 230 is a rectangular upper plate, and a protrusion protruding inward in a curved shape is formed at the center of both ends of the rectangular shape in a long side direction thereof to support the blister.

The blister seating portion 230 may be formed to correspond to the arrangement of blisters. For example, as illustrated in FIG. 10, an intermediate wall may be longitudinally formed at the center of the container main body to allow two blisters to be seated in parallel, and an upper portion of the intermediate wall may constitute a portion of the blister seating portion 230.

A plurality of support portions 240 are disposed to be aligned with each other to correspond to a contents arrangement structure of the blisters inside the container main body 200. In order to facilitate pressing the content in the blister by hand or tool so that the content falls to the container main body as illustrated in FIG. 2, the plurality of support portions 240 are formed at intermediate positions between positions where the contents are disposed. The plurality of support portions 240 may be integrally formed with the container main body but are not limited thereto.

The plurality of support portions 240 each formed in the shape of a pillar are formed to extend from a bottom of the container main body so as to be positioned at bottom portions between the contents arranged in the blisters. Due to such a structure, when the contents in the blisters are pressed downward, the plurality of support portions support the blisters from below and facilitate extrusion and falling of the contents.

Meanwhile, a plurality of seating protrusions 250 are formed to be bent on the blister seating portion 230 so that the blisters are reliably seated, and edge portions of the blisters are fitted between the seating protrusions 250 and the blister seating portion 230. More specifically, the blisters are fitted between the seating protrusions 250 and the blister seating portion 230 by sliding along an upper portion of the blister seating portion 230 on one side of the container main body.

When a user presses the content in the stably fitted blister, the support portions 240 at both sides of the bottom of the content support portions of the blister at both sides of the pressed content, and thus the content is effectively pressed. Meanwhile, the curved protrusions at both ends of the support portions also support the blister from both ends to support the blister at the time the content positioned at an edge is extruded, and thus the extrusion of the content is facilitated. Such a structure can simultaneously facilitate the extrusion of the content and allow the content to fall into the container main body and move.

The extruded content may fall to the bottom of the container main body, and the container main body may accommodate the content discharged from the blister and deliver the content to the passing portion.

### INDUSTRIAL APPLICABILITY

The present disclosure is for allowing a drug to be taken in fixed amounts and may be used in the industrial field of drug-related devices.

1. A contents accommodation device comprising:
a blister seating portion for seating a blister pack in which contents are packed;
a container main body configured to accommodate the blister seating portion in an upper portion thereof and having a space at a lower portion to accommodate contents that fall after being extruded from the blister pack; and
a passing portion configured to discharge the contents moving thereto from the container main body to an outside,
wherein the passing portion includes a passage portion configured to serve as a movement passage of the contents and a detecting portion configured to detect movement of the contents,
an opening is formed at one end of the passage portion, and
the contents are discharged to the outside through the opening at the one end of the passage portion after entering the passage portion from the container main body.

2. The contents accommodation device of claim 1, wherein:
the passing portion is connected to one side of the container main body;
the passage portion forms a movement path to discharge the contents in fixed amounts and communicates via an inlet with the space of the container main body that accommodates the contents;
the container main body includes a guide passage disposed adjacent to the inlet to guide the contents toward the passage portion; and
the contents enter the passage portion along the guide passage.

3. The contents accommodation device of claim wherein:
the detecting portion includes a light emitting portion and a light receiving portion disposed to face the light emitting portion;
light radiated from the light emitting portion is detected by the light receiving portion after passing through the path along which the contents move to be discharged, and whether the contents are discharged is determined by a quantity of light detected by the light receiving portion; and
the contents are determined as having been discharged in a case in which the quantity of light detected by the light receiving portion that belongs to a standard light quantity range becomes a predetermined reference light quantity or less and then rises back into the standard light quantity range.

4. The contents accommodation device of claim 1, wherein:
the detecting portion includes a light emitting portion disposed at one side of the passage portion and a light receiving portion disposed at the other side of the passage portion to face the light emitting portion;
the light receiving portion includes a first light receiving portion disposed at an upstream side of the path along which the contents move to be discharged and a second light receiving portion disposed at a downstream side of the path;
the light emitting portion includes one or more light emitting portions disposed to face the light receiving portion; and
whether the contents are discharged is determined based on a light quantity change pattern by time that is detected by each of the first light receiving portion and the second light receiving portion.

5. The contents accommodation device of claim 4, wherein the contents are determined as not having been discharged in a case in which the contents are determined as having passed through an area of the second light receiving portion prior to passing through an area of the first light receiving portion.

6. The contents accommodation device of claim 4, wherein a first point in time, at which a quantity of light detected by the first light receiving portion that belongs to a first standard light quantity range becomes a first reference light quantity or less and then rises back into the first standard light quantity range, and a second point in time, at which a quantity of light detected by the second light receiving portion that belongs to a second standard light quantity range becomes a second reference light quantity or less and then rises back into the second standard light quantity range, are compared, and the contents are determined as having been discharged in a case in which the first point in time is the same as or earlier than the second point in time.

7. The contents accommodation device of claim 1, wherein:
the blister seating portion is installed between a container lid and the container main body and seated on an upper end of the container main body and includes a seating plate and a seating lid coupled to an upper portion of the seating plate;
the seating lid is hinge-coupled to one side of the seating plate;
the seating plate supports the blister pack from below;
the seating lid is a plate in which a plurality of through-holes aligned with each other are formed; and
contents accommodating portions of the blister pack seated on the seating plate are each disposed in the through-holes.

8. The contents accommodation device of claim 2, wherein:
the guide passage is formed to be bent from the inlet;
the passage portion is formed to have a slope from the space; and
a width of the passage portion is formed to allow only one content to pass at a time.

9. The contents accommodation device of claim 1, wherein a signal detected by the detecting portion is processed by an information processing device included in the passing device or is processed by a separate external device.

10. The contents accommodation device of claim 1, wherein:
a plurality of support portions are disposed to be aligned with each other inside the container main body to correspond to a contents arrangement structure of the blister pack; and
upon a user pressing one content, the plurality of support portions support portions of the blister pack at both sides of the pressed content from below to facilitate extrusion of the content.

11. A method of determining whether contents are discharged based on a quantity of light detected by a detecting portion of the contents accommodation device of claim 1, wherein the detecting portion includes a light emitting portion and a light receiving portion disposed to face the light emitting portion, the light receiving portion includes a first light receiving portion disposed at an upstream side of a path along which the contents move to be discharged and a second light receiving portion disposed at a downstream side of the path, and the light emitting portion includes one or more light emitting portions disposed to face the light receiving portion, the method comprising:
a detection step in which both the first light receiving portion and the second light receiving portion detect a quantity of light that deviates from a standard light quantity range and is lower than a reference light quantity;
a detection step in which the first light receiving portion detects a quantity of light that deviates from a first reference light quantity and belongs to a first standard light quantity range, and the second light receiving portion detects a quantity of light that deviates from a second reference light quantity and belongs to a second standard light quantity range;
a comparison step in which a first point in time, at which the quantity of light detected by the first light receiving portion that belongs to the first standard light quantity range becomes the first reference light quantity or less and then rises back into the first standard light quantity range, and a second point in time, at which the quantity of light detected by the second light receiving portion that belongs to the second standard light quantity range becomes the second reference light quantity or less and then rises back into the second standard light quantity range, are compared; and
a determination step in which the contents are determined as having been discharged in a case in which the first point in time is the same as or earlier than the second point in time.
